(19) United States
(12) Patent Application Publication
(10) Pub. No.: US 2017/0239179 A1
(43) Pub. Date: Aug. 24, 2017

(54) Flavored Water Composition For Pets

(71) Applicant: Melanie Revucky, Downingtown, PA (US)

(72) Inventor: Melanie Revucky, Downingtown, PA (US)

(21) Appl. No.: 15/048,293

(22) Filed: Feb. 19, 2016

(51) Int. Cl. A61K 9/00; A61K 31/202; A61K 31/201; A61K 31/59; A61K 31/122; A23K 50/48; A61K 45/06; A23K 20/158; A23K 20/174; A23K 20/179; A23K 20/105; A61K 35/60; A61K 47/12

(52) U.S. Cl. CPC A61K 9/0095; A61K 35/60; A61K 31/202; A61K 31/201; A61K 31/59; A61K 31/122; A61K 47/12; A61K 45/06; A23K 20/158; A23K 20/174; A23K 20/179; A23K 20/105; A23K 50/48

(57) ABSTRACT

A flavored water composition for pets is disclosed. The disclosed flavored water composition for pets includes water and an oil-based nutrient.

### FIELD OF THE INVENTION

The present invention relates to a water composition and, more particularly, to a flavored water composition for pets.

### BACKGROUND

Numerous food products promoting pet health are known, including various formulas of dry and wet food, vitamins, and other supplements. These solid products, however, are often difficult to administer to pets, undermining the effectiveness of the health-improving elements contained within.

In addition to the nutrients provided by food, clean water is a critical and substantial element of a healthy pet's diet. Many pet owners who feed their pets health-promoting solid foods pay little attention to the freshness and quality of their pet's water. Unclean water, however, can harbor harmful bacteria and independently diminish pet health.

### SUMMARY

An object of the invention is to provide a flavored water composition for pets that is clean and provides an additional source of healthy nutrients. The disclosed flavored water composition for pets includes water and an oil-based nutrient.

### DETAILED DESCRIPTION OF EMBODIMENT(S)

The invention is explained in greater detail below with reference to embodiments of a flavored water composition for pets. This invention may, however, be embodied in many different forms, and should not be construed as limited to the embodiments set forth herein; rather, these embodiments are provided so that this disclosure will be thorough and complete and still fully convey the scope of the invention to those skilled in the art.

The flavored water composition for pets according to the present invention generally comprises an emulsion of water and an oil-based nutrient. The major components of the invention will now be described in greater detail.

The water in the composition may be purified water, distilled water, spring water, mineral water, or other types of water known to those with ordinary skill in the art. The composition may include water between 90% and 99.99% of the composition by volume, and in an optional further embodiment of the invention, the composition may include water between 99% and 99.99% of the composition by volume.

The oil-based nutrient in the composition may be a fish oil, or other types of oils including vitamins or minerals known to those with ordinary skill in the art. The composition may include an oil-based nutrient between .01% and 10% of the composition by volume, and in an optional further embodiment of the invention, the composition may include an oil-based nutrient between .01% and 1% of the composition by volume.

The oil-based nutrient may be mackerel oil, herring oil, tuna oil, halibut oil, salmon oil, Alaskan salmon oil, and cod liver oil, or other fish oils known to those with ordinary skill in the art. The oil-based nutrient may contain omega-3 fatty acids, including alpha-linolenic acid (ALA), eicosapentaenoic acid (EPA), and docosahexaenoic acid (DHA).

As would be understood by one with ordinary skill in the art, the composition and/or the oil-based nutrient may also contain, together or in any combination, calcium, iron, vitamin A, vitamin B1, vitamin B2, vitamin B3, vitamin C, vitamin D, vitamin E, vitamin K, and astaxanthin.

In another embodiment, the flavored water composition may contain a water-soluble preservative. The water-soluble preservative may be sodium benzoate, potassium benzoate, potassium sorbate, citric acid, or other food-safe, water-soluble preservatives known to those with ordinary skill in the art.

Advantageously, the flavored water composition for pets according to the present invention promotes the consumption of water due to the improved flavor while also providing an additional source of nutrients to the pet. The increased consumption of water leads to myriad physical benefits alone. Additionally, the added nutrients can improve cardiovascular functioning, cognitive reflexes, eye, skin and coat health, immune function, can reduce inflammation, and can reduce joint pain, among other health benefits. The liquid source of nutrients provided by the flavored water composition is also easier for pets to consume, reducing the pet owner's time and effort necessary to promote their pet's health.

The foregoing illustrates some of the possibilities for practicing the invention. Many other embodiments are possible within the scope and spirit of the invention. The disclosed invention utilizes the above identified components, as a composition, in order to promote consumption of nutrients for a particular purpose. Therefore, more or less of the aforementioned components can be used to conform to that particular purpose. It is, therefore, intended that the foregoing description be regarded as illustrative rather than limiting, and that the scope of the invention is given by the appended claims together with their full range of equivalents.

1. A flavored water composition for pets, comprising:
an improved drinking water comprising between 90% and 99.99% of the composition by volume; and
a fish oil-based nutrient additive emulsified with the water, the fish oil-based nutrient derived from salmon comprising between .01% and 10% of the composition by volume, and excluding a lipophilic amphilphilic additive, an extract of rosemary, or an extract of oregano.

2. (canceled)

3. (canceled)

4. The flavored water composition for pets of claim 1, wherein the improved drinking water is between 99% and 99.99% of the composition by volume.

5. The flavored water composition for pets of claim 4, wherein the fish oil-based nutrient is derived from salmon is between .01% and 1% of the composition by volume.

6. (canceled)

7. (canceled)

8. The flavored water composition for pets of claim 1, wherein the fish oil-based nutrient derived from salmon includes Alaskan salmon oil.

9. The flavored water composition for pets of claim 8, wherein the fish oil-based nutrient derived from salmon includes eicosapentaenoic acid and docosahexaenoic acid.

10. The flavored water composition for pets of claim 9, wherein the fish oil-based nutrient derived from salmon includes alpha-linolenic acid.

11. The flavored water composition for pets of claim 10, wherein the fish oil-based nutrient derived from salmon includes vitamin D.

12. The flavored water composition for pets of claim 11, wherein the fish oil-based nutrient derived from salmon includes astaxanthin.

13. The flavored water composition for pets of claim 1, wherein the improved drinking water is purified water.

14. The flavored water composition for pets of claim 13, further comprising a water-soluble preservative.

15. The flavored water composition for pets of claim 14, wherein the water-soluble preservative is citric acid.

16. The flavored water composition for pets of claim 1, further comprising one or more vitamins from a group consisting of calcium, iron, vitamin A, vitamin B1, vitamin B2, vitamin B3, vitamin C, vitamin D, vitamin E, or vitamin K.

17. The flavored water composition for pets of claim 16, further comprising astaxanthin.

18. A method for promoting consumption of water by a pet comprising:
preparing an emulsion of improved drinking water and a fish oil-based nutrient additive, the improved drinking water comprising between 90% and 99.99% of the emulsion by volume, the fish oil-based nutrient derived from Alaskan salmon and comprising between .01% and 10% of the emulsion by volume from salmon, and the emulsion excluding a lipophilic amphilphilic additive, an extract of rosemary, or an extract of oregano; and providing a pet access to the emulsion.
